SEQUENCE LISTING (8 sequences)

SEQ ID NO: 1 - DNA, 101 bp, Rattus rattus
actttctctatagctcctggtaagtaaatttctttctccaatactttttgagttaaatgttttagtttatgtgggggtttagttatgttggttggttgtag

SEQ ID NO: 2 - DNA, 299 bp, Rattus rattus
atttttaaaaagcagccggggcctggggtttctacccgtgtaccaggggccctctggcccagagctgaccaaatctggctccatggagcacacagaggctttgatcagggacagtaatcctctgcaacatcaggaatggctgaatgcacaggatttaccaagcctcagccaaagcatcccgtggcctgatgtctcggagcaaccctgtccacacgaggaaaggtcaggcctgctcaacatgaccaagattgctcaaggagggcgcaaactcaggaagagccggggccctgcttgggtag

SEQ ID NO: 3 - DNA, 560 bp, Rattus rattus
gactttattcacaatagagaaattttacaaatataatttttaaaaattatgtgtcaatctattatgttttccgtaacatcagagatttatataaagttggaaacaacagaatgcacttatgaacaaatcaaaaacaatgtttaaattggatggatacacacgacagagaagtcactgagttctctaaatgagcacacaacttataggtgtatattaactgcacaaagtatccaaaacatgtttgtaacacaaaatcgggtgctactttaactgctcacctttaagggcgtggatcatacatgtaagtcaaattgcacagctttgttggaaatgaatgactcgtcatctatttggagacttccgttgcttaaaattgacacaaaagcctaatcaattacgctactataaaatttgtctcttatctcgtttaaatttttggtgttctgtgatctggcattaaaaaacagtccaagttttaaaacagaaaacattgctcgccagttggagagtagctcgtggttcggcttcctccctgctcgaaccggaacaaacgctacagt

SEQ ID NO: 4 - DNA, 310 bp, Rattus rattus
acattcattattaaatgtgataatagaggtagaggtataaataatatgaaggggtgagggaaccagttctacccggtttgttttgaatgcttaaattatgtaatttaaatagataatctttacttatgtaggtcttttggaaataactttataaatttaacacagaggactactactaaacgtgagaggtatgataatcggcatggaagttgggctggttgaccaccaaagttcaattcttaaagacatcttaatcctgaatataaaaatgcctttgtgggtttagaattagaatttaattttggcattt

SEQ ID NO: 5 - DNA, 136 bp, Rattus rattus
actgcatgatgggttttattgagaccaggggacagtgtgacactcaggggttttccttcataacttcttttatccaggaggtgaacttaataagtttggtgtagatggctggcatgttggttttggcgcatgatag

SEQ ID NO: 6 - DNA, 347 bp, Rattus rattus
ctatcatgcgtgtagtcttggtgccctggccgagttagaagccagctgagatagcttgcagcatctcttctagtttgagtgatgatgtaatgaggaaaatctagtaggtagaaagagttcaggaagaaggaaaccctcctctgcctttgaaaagaggctctgcaggagcatcacgcccttcacagagaagagtgtagactggctttccactagtgttgaacctacactcttcggtgggttaacagtcatgtgctcgccatcagagcctttttgcatgcagtggtgggctctcccggtttatcccacctcccacaggtgattaaaccacagccctgtaaaaaaaaaaa

SEQ ID NO: 7 - DNA, 513 bp, Rattus rattus
ttacccacagtgcattataacaaaggagatgctaaagtcagtttttcatgtttgtggtttttctgaaacatcattcatttaaacaattcaaatatatgttcaaaataagaagttgtttataaaaggattgtgtgtgccatgtggcttttgacccgtgctattataaatgttgccataaatactctctataagaaacagtccttaagtagatttggtggcacacatctttaatcccagcacttgggaagcagagacaggtggatctctgtgagtttaagaccaacctggtctataaagtgagttccaggacagccagggttgttaaacatagagaaactctggggcgatggggaggggtctcgtcaaacatgaaatttattagaaaattggtcggattaagctatgtctagtatcaactaatatggaatcttgtataatctgtgttacattggatttgtctcagaactaattgtttcataataaactatgccttggccaccacgaaaaaaaaaa

SEQ ID NO: 8 - DNA, 313 bp, Rattus rattus
aggctagggctagttctgcggaccctctcggagagaggaataaggttgaactgcctgtccggttctccttcccctattcccagatgcaggtggaagcctccctctagtccttccccctaaccgcgacgaagaccttggctaacacttgctcctttcgcacaccatagaaaatgcagtgcagacaaacacagcctcgtcaggcgcttgaggagcgaagtccaatctgggtcggcacctgcaccaggtctttgcgcacctggtcagaagaccggcacccaatagttgcttattaaactctacgtttgtcccgaaa

(19) United States
(12) Patent Application Publication
(10) Pub. No.: US 2008/0044403 A1
(43) Pub. Date: Feb. 21, 2008

(54) Immune Markers Used for Diagnosis and Therapy in Connection With Transplant Reactions

(76) Inventors: Birgit Sawitzki, Berlin (DE); Hans-Dieter Volk, Berlin (DE); Kathryn Wood, Oxfordshire (GB); Manfred Lehmann, Rostock (DE)

Correspondence Address: WOOD, HERRON & EVANS, LLP, 2700 CAREW TOWER, 441 VINE STREET, CINCINNATI, OH, 45202, US

(21) Appl. No.: 10/525,180

(22) PCT Filed: Aug. 22, 2003
(86) PCT No.: PCT/EP03/09355

§ 371 Date: Sep. 27, 2006

(30) Foreign Application Priority Data
Aug. 22, 2002 (DE) 102 38 922.5

(51) Int. Cl. A61K 31/70; A61K 31/395; A61P 43/00; C07H 21/04; C07K 16/00; C12N 15/00; C12N 5/00; C12Q 1/68

(52) U.S. Cl. 424/130.1; 424/93.7; 435/320.1; 435/325; 435/6; 514/12; 514/44; 530/350; 530/387.1; 536/22.1; 536/24.5

(57) ABSTRACT

The invention relates to immune markers for detecting inflammations, immune reactions, and particularly transplant tolerance or transplant reactions, a method for detecting transplant reactions or tolerance, and the use of immune markers for medical prophylaxis, clinical monitoring of progress, transplant follow-up treatment, clinical diagnosis and/or therapy in connection with cell transplants, tissue transplants, or organ transplants.

The invention relates to nucleic acid molecules as immune markers for the detection of graft reactions, a method for the detection of graft reactions, and the use of immune markers for medical prophylaxis, clinical follow-up, graft follow-up treatment, clinical diagnostics and/or therapy in connection with cell, tissue and organ transplantations, where graft reactions may be tolerance, rejection crisis or rejection.

For an organ transplantation to be successful, it is necessary that the donor organ have as high as possible a histological correspondence with the receptor tissue. This correspondence is determined, inter alia, by the HLA (human leukocyte antigen) system. This is a complex system of tissue antigens which occur on almost all cells. This system plays an important physiological role in immunological defense reactions (recognition of “self” and “non-self”). Therefore, prior to each transplantation, a so-called tissue typing is performed with the organ donor and receptor in order to ensure as high as possible an HLA compatibility.

Due to the extremely high genetic polymorphism, there are an extraordinarily high number of different HLA molecules. A complete correspondence is observed exclusively with identical twins, HLA molecules being unique for each human otherwise.

However, there is a problem in that a rejection reaction against the grafted organ cannot be excluded despite a substantial HLA correspondence between the receptor and donor. Despite these difficulties, transplantation is the therapy of choice for irreversible organ failure.

The increasing demand for organ transplantations together with the reduced supply of organs as well as the problems described above necessitate an optimization of the known therapies. By introducing new improved immunosuppressants, the one-year survival rate could be increased to 90%. However, to date, it has not been possible to satisfactorily improve the long-term graft survival rates. Despite of modern immunosuppressants, chronic graft dysfunctions still develop in the majority of patients. Clinical and subclinical acute reactions, even if treated successfully at first with a rejection therapy, represent the greatest independent risk factor of the development of these late graft losses. Therefore, efficient diagnostics and/or an efficient therapy of such processes, which mainly have a subclinical course, is of great importance.

Therefore, induction of a long-term graft acceptance, preferably without medicaments, without inversely affecting the organ, tissue or cell functions and the immune capacity of the graft receptor is of great importance. Clinically, tolerance is defined as a permanent survival of the graft with a normal organ function with absence of acute and chronic rejection reactions and preservation of the antimicrobial immunoreactivity.

New strategies for the induction of a graft tolerance consist in the application of immunoregulatory proteins or induction of chimerism. The immunoregulatory proteins may be either antibodies which cause depletion of the donor-reactive T cells, e.g., anti-CD3 immunotoxin, or antibodies and proteins which affect the activation of the donor-reactive T cells, e.g., anti-CD4 antibodies, anti-CD40L antibodies or CTLA4-Ig. Chimerism means the parallel presence of blood leukocytes of the donor and receptor achieved by non-myelo-ablative methods for the transplantation of stem cells of the donor.

To date, after transplantation, a permanent supervision of the condition of the graft has been performed by recurring to the function of the graft as a measure of its condition, for example, by determining the serum creatinine level in the case of a kidney graft. In addition, bioptates are removed and their histology evaluated according to the “Banff score”. Thus, it can be evaluated whether changes associated with acute rejection, i.e., infiltration of mononuclear cells, or chronic rejections (vasculopathy) can be detected.

Clinically manifest rejections are defined by a functional deterioration of the organs, such as cardiac function, serum creatinine, lung function or others. Unfortunately, these functional deteriorations are at the end of an effector chain. Early diagnostics already of processes with a subclinical course would be very helpful. Also, functional deterioration is not always due to rejection; there are other causes, such as toxicity and infections, which must be delimited by differential diagnostics, which currently demands a lot of time and is often very difficult. To date, subclinically proceeding reactions could be evaluated reliably, at least within certain limits, only by protocol biopsies and conventional histology. However, those infiltrates which possibly have a protective effect on the graft, as could be shown in animal experiments, are also evaluated as negative. For kidney grafts, it has been proven that molecular-biological examinations in the urine can reflect a clinically manifest rejection, but there are no studies relating to the subclinical range of rejections. Thus, there is a great demand for markers for the monitoring of grafts (bioptates, urine, lavage, blood etc.) in order to detect undesirable immune reactions against the graft in due time, i.e., possibly before the organ is damaged, and with certainty in terms of differential diagnostics as a delimitation against other processes which adversely affect organ function.

Due to the side effects of the chronic application of the currently usual multiple immunosuppressant schemes, it is tried again and again to discontinue the administration of one or more of the immunosuppressant components when the function is going well; disadvantageously, this approach is always endangered by the occurrence of accelerated rejection processes, sometimes only years after the discontinuation. There is a complete lack of parameters which could reliably optimize such an approach on an individual basis. An improvement of the previous strategies by introducing tolerance-induced protocols would revolutionize the therapy due to less side effects and a lesser cost. However, transfer of the above mentioned tolerance-induced therapies to humans involve quite a few risks, since those in whom the therapy fails must be identified in due time after the discontinuation of the tolerance induction therapy in order to prevent irreversible damage to the graft by rejection. Even in animal models, it is never observed that 100% of the receptors become tolerant.

The previous methods have a disadvantage in that no decision on the safe discontinuation of an immunosuppressive therapy can be made without risking the occurrence of rejection crises.

Another drawback is the fact that the known methods do not allow the prediction of rejection crises during or after the treatment, also after conventional therapies, before a deterioration of the graft function occurs. The diagnostic means and methods which have been available to date can be employed for evaluating a tolerance-induced therapy only in a limited way. Evaluation of the therapy in terms of the function of the graft, e.g., by means of serum creatinine, is too late because, when an increase of serum creatinine can be detected, the grafted organ, for example, the kidney, has already been damaged. With respect to the tolerance-induced therapies, a significant increase of serum creatinine means failure of the therapy and, for the patient, probably change to conventional immunosuppressants with the known side effects. The known analysis of a biopsy is also helpful only in a qualified way, since many tolerance-induced therapies, such as with anti-CD4 antibodies, prevent the infiltration of mononuclear cells into the graft only in a qualified way. Within the scope of the previous methods and procedures, this would be considered an acute rejection crisis, and the patient would be treated with highly dosed conventional immunosuppressants. However, the highly dosed administration of conventional immunosuppressants can have additional negative effects on the success of the tolerance-induced therapy, which would also mean failure of the therapy. But even for the conventional immunosuppressions, improved diagnostic agents and methods would be helpful in terms of the recognition of clinical and subclinical acute rejection crises and beginning chronic rejection processes. This would allow, inter alia, to vary a therapy before a damage to the graft is detectable, e.g., by an increase of serum creatinine. In addition, a deterioration of organ function may also be caused as a side effect of a high-dose immunosuppressant therapy, or by infections in the graft, which cannot be detected wither by known methods.

Therefore, it is the object of the invention to provide efficient and reliable immune markers which enable a certain and fast prediction of the risk of a graft rejection or the absence thereof, as a form of tolerance, in medical prophylaxis, clinical follow-up or graft follow-up treatment.

The present invention achieves this object by providing an isolated nucleic acid molecule selected from the group consisting of:
a) a nucleic acid molecule comprising a nucleotide sequence selected from the group consisting of SEQ ID Nos. 1 to 8 or their complementary nucleotide sequences;
b) a nucleic acid molecule which will hybridize with a nucleotide sequence according to a) under stringent conditions;
c) a nucleic acid molecule comprising a nucleotide sequence which has sufficient homology with a nucleotide sequence according to a) or b) to be a functional analogue thereof;
d) a nucleic acid molecule which exhibits a genetic code degeneration relationship with respect to a nucleotide sequence according to any of a) to c); and
e) a nucleic acid molecule according to any nucleotide sequence of a) to d) which has been modified by deletions, additions, substitutions, translocations, inversions and/or insertions and is a functional analogue of a nucleotide sequence according to any of a) to d).

It has been surprising that the nucleic acid molecules according to the invention are associated with inflammations, especially chronic inflammations, auto-immune diseases, lesions, general wounds and graft reactions, especially graft rejections or other graft dysfunctions as well as the absence thereof as a form of graft tolerance.

In a preferred embodiment of the invention, the nucleic acid molecule which has a sufficient homology to be a functional analogue of a nucleotide sequence selected from the group consisting of SEQ ID Nos. 1 to 8 or their complementary nucleotide sequences has at least 40% homology. According to the invention, “to be a functional analogue” of the nucleotide sequences mentioned or of the sequences which will hybridize with these nucleotide sequences means that for graft reactions the homologues exhibit a behavior which allows to draw conclusions on the graft and its relation to the receptor organism.

According to the invention, functionally analogous sequences are all those sequences which can be identified by the skilled person as having the same effect. For example, it is possible that the skilled person identifies nucleic acid molecules according to the invention in different test animals, such as rats or rabbits, and thus is able to identify functionally analogous structures in other organisms, such as chimpanzees or dogs, due to homology studies. Of course, it is also possible for the skilled person to detect analogues and homologues in human patients in homology or analogy studies due to his knowledge of the nucleic acid molecules found in mice or rats. Further, it is possible for nucleic acid molecules according to the invention isolated in the human field to be detected by the skilled person in specific test animals with which particular graft reactions can be examined, such as in pigs or even in invertebrate organisms, such as nematodes or other organisms which may be recurred to for specific items of graft biology.

In another advantageous embodiment of the invention, the nucleic acid molecule has at least 60%, preferably 70%, more preferably 80% and still more preferably 90% homology with a nucleic acid molecule comprising a nucleotide sequence selected from the group consisting of SEQ ID Nos. 1 to 8 or their complementary nucleotide sequences, said nucleic acid molecule having a biological activity similar to the sequences shown under SEQ ID Nos. 1 to 8 or their complementary sequences.

In another preferred embodiment of the invention, the nucleic acid molecule is a genomic DNA, a cDNA and/or an RNA. More preferably, the nucleic acid molecule is an mRNA.

The invention also relates to a vector which comprises a nucleic acid molecule according to the invention. Further, the invention also relates to a host cell which comprises the vector according to the invention. The invention also relates to a polypeptide which is encoded by a nucleic acid molecule according to the invention.

The invention also relates to a recognition molecule which is directed against the nucleic acid molecule, the vector, the host cell and/or the polypeptide. According to the invention, recognition substances are molecules which are able to interact with the structures mentioned, such as nucleic acid molecules or sequences, vectors, host cells and/or polypeptides or their fragments, in particular, to interact in such a way that detection of these structures becomes possible. In particular, the recognition substances may be specific nucleic acids which bind to the nucleic acid molecules mentioned, but also antibodies, fluorescent markers, labeled carbohydrates or lipids, antisense constructs, cDNA or mRNA molecules, or their fragments. Of course, it is also possible that the recognition substances are not proteins or nucleic acids or antibodies, but antibodies directed against them. In such a case, the recognition substances may be secondary antibodies, in particular.

In a particular embodiment of the invention, the recognition molecules include an antibody, an antibody fragment and/or an antisense construct, especially an RNA interference molecule.

The auto-antibodies according to the invention specifically bind the polypeptides according to the invention. The antibodies may also be modified antibodies (e.g., oligomers, reduced, oxidized and labeled antibodies). The term “antibody” as used in the present description comprises both intact molecules and auto-antibody fragments, such as Fab, F(ab′)₂ and Fv which can bind specific epitope determinants of the polypeptides. In these fragments, the capability of the antibody of selectively binding its antigen or receptor is partially retained, the fragments being defined as follows:
(1) Fab, the fragment which contains a monovalent antigen-binding fragment of an antibody molecule, can be produced by cleavage of a whole antibody with the enzyme papain to obtain an intact light chain and a part of a heavy chain;
(2) the Fab′ fragment of an antibody molecule can be obtained by treating a whole antibody with pepsin followed by reduction to obtain an intact light chain and a part of a heavy chain; two Fab′ fragments are obtained per antibody molecule;
(3) F(ab′)₂, the fragment of the antibody which can be obtained by treating a whole antibody with the enzyme pepsin without a subsequent reduction; F(ab′)₂ is a dimer of two Fab′ fragments connected by two disulfide bridges;
(4) Fv, defined as a genetically engineered fragment which contains the variable region of the light chain and the variable region of the heavy chain and is expressed in the form of two chains; and
(5) single-chain antibody (“SCA”), defined as a genetically engineered molecule which contains the variable region of the light chain and the variable region of the heavy chain connected by a suitable polypeptide linker to form a genetically fused single-chain molecule.

The term “epitope” as used in the present invention means any antigen determinant on the polypeptide. Epitope determinants normally consist of chemically active surface groups of molecules, such as amino acids or sugar side chains, and normally possess both specific characteristics of the three-dimensional structure and specific charge characteristics.

The invention also relates to vaccines which comprise the nucleic acid molecule, the vector, the host cell, the polypeptide and/or the recognition molecule, optionally with a pharmaceutically acceptable carrier. The pharmaceutically acceptable carrier includes per se known pharmaceutical auxiliary agents and/or additives. These additives and carrier substances which are per se known to the skilled person may also be liposomes or structures known in genetic engineering, or solutions and/or buffer mixtures, or other substances from the field of galenics.

The invention also relates to a method for the detection of graft reactions in a sample from a patient, wherein a level of at least one nucleic acid molecule selected from the group consisting of:
a) a nucleic acid molecule comprising a nucleotide sequence selected from the group consisting of SEQ ID Nos. 1 to 8 or their complementary nucleotide sequences;
b) a nucleic acid molecule which will hybridize with a nucleotide sequence according to a) under stringent conditions;
c) a nucleic acid molecule comprising a nucleotide sequence which has sufficient homology with a nucleotide sequence according to a) or b) to be a functional analogue thereof;
d) a nucleic acid molecule which exhibits a genetic code degeneration relationship with respect to a nucleotide sequence according to any of a) to c); and
e) a nucleic acid molecule according to any nucleotide sequence of a) to d) which has been modified by deletions, additions, substitutions, translocations, inversions and/or insertions and is a functional analogue of a nucleotide sequence according to any of a) to d);
   is determined in the sample, and the level is compared with a control level of a comparative sample from a healthy patient, wherein the graft reactions, which also include the absence thereof as a tolerance, are detected by a modified level in the sample as compared to the control level.

Accordingly, “graft reaction” according to the invention means any physiological and pathophysiological interaction of the graft with the receptor organism, but also any isolated reaction within the graft. Therefore, according to the invention, the graft reaction can be a tolerance or a rejection of the graft. Accordingly, a graft reaction according to the invention also includes a non-pathological, i.e., normal or healthy, condition in which the graft may be by itself and with respect to the receptor organism. A “sample” within the meaning of the invention is the designation of a biological item removed by sampling or a part or small amount thereof the quality of which is to be tested chemically, biologically, clinically or in a similar way. In particular, the sampling from the patient or from humoral or cellular components obtained from the patient is performed in such a way that the fraction removed corresponds to an average of the whole amount. The features established by examining the sample serve for evaluating the amount covered by the sample which allows conclusions to be drawn on the entire amount, for example, a whole grafted organ, such as liver, spleen, blood, or else non-transplanted components, such as the immune system. For the examination, the samples may first be pretreated by mixing, dividing, comminuting, addition of enzymes or markers or otherwise. Various possibilities for pre-treatment of the samples are known to the skilled person. Of course, it may also be provided that the sample is removed in such a way that it does not correspond to any average of the entire amount. A sample may include all biological and non-biological materials, such as biological tissues and fluids, such as blood, lymph, urine, liquor and others.

A graft within the meaning of the invention is an organ, tissue or cell or accumulation of cells which has been grafted or is to be grafted. However, according to the invention, grafts may also be certain implants consisting of materials or parts which are introduced into a body for a limited period of time or for a lifetime for fulfilling particular replacement functions. For example, the implants may consist of inorganic matter which is coated with organic substances, such as cartilage or bone cells.

According to the invention, “graft rejection” means the induction of an immune response by the receptor to the graft, an immune response of the receptor being a specific protection or defense reaction of the body against the antigens or other structures of the graft.

According to the invention, a “patient” means any organism which comprises a graft, especially a human organism. A “healthy patient” according to the invention is a patient whose condition allows him to be used as a reference for the present method. “Healthy” according to the invention need not mean the complete absence of diseases, grafts or pathogenic alterations. The “healthy patient” either represents an individual patient or an average set of patients who can serve as a comparative group in such a way that a change of the level of the mentioned nucleic acid molecules or of the structures for which they code or the recognition substances can be determined. “Modification” of the level as compared to the control level means that the nucleic acid molecules mentioned or the above mentioned immune markers, such as the peptides or the recognition substances, in particular, exhibit detectable changes in their concentration or activity as a protein, as a nucleic acid molecule or as an antibody as compared to the control level.

In a particular embodiment of the invention, the graft is selected from the group consisting of lung, spleen, heart, liver, pancreas and/or tissues, especially islets, aortas, cartilage. Of course, it is possible that the respective organs or tissue structures can be grafted alone or in combination.

In another preferred embodiment of the invention, the level is determined as a DNA or RNA concentration, gene expression, number of copies of a nucleic acid, peptide concentration, peptide activity and/or as a concentration of isoforms. Advantageously, the skilled person has different possibilities to chose from for determining the level of at least one nucleic acid molecule. For example, one possibility is to determine the concentration of the peptides which are encoded by the nucleic acid molecule with spectrographic methods. However, it is also possible to determine the level on the RNA or DNA, especially mRNA and/or cDNA, levels or, for example, through the activity of the proteins and/or peptides encoded thereby or their fragments. Of course, it is possible that the level is determined only in the graft or in fragments thereof inside or outside the body, or that it is detected in the surrounding tissue or body fluids, or in biopsy materials, or in fluids such as urine, lymph or blood.

In another preferred embodiment of the invention, the level is determined as an mRNA concentration.

In another preferred embodiment of the invention, the graft reaction is a rejection crisis, a rejection reaction, a course of a rejection, a tolerance reaction and/or a course of a tolerance which is detected by the method according to the invention. The course of a rejection and the rejection reaction can be, for example, clinical or subclinical. A tolerance according to the invention is, for example, a long lasting normal function of the grafted organ without an increase of the serum creatinine or proteinuria for more than 100 days, preferably 200 days, more preferably 300 days.

In another preferred embodiment of the invention, the rejection reaction, the course of a rejection and/or the rejection crisis is detected by a reduced level of a nucleic acid molecule selected from the group consisting of:
a) a nucleic acid molecule comprising a nucleotide sequence selected from the group consisting of SEQ ID No. 3 and SEQ ID No. 7 or their complementary nucleotide sequences;
b) a nucleic acid molecule which will hybridize with a nucleotide sequence according to a) under stringent conditions;
c) a nucleic acid molecule comprising a nucleotide sequence which has sufficient homology with a nucleotide sequence according to a) or b) to be a functional analogue thereof;
d) a nucleic acid molecule which exhibits a genetic code degeneration relationship with respect to a nucleotide sequence according to any of a) to c); and
e) a nucleic acid molecule according to any nucleotide sequence of a) to d) which has been modified by deletions, additions, substitutions, translocations, inversions and/or insertions and is a functional analogue of a nucleotide sequence according to any of a) to d).

A course of a rejection may be, for example, the course of a rejection with or without the administration of medicaments, wherein such medicaments may be immunosuppressant substances, for example. Advantageously, from the reduced level of the nucleotide sequences or their complementary nucleotide sequences or nucleic acid molecules which will hybridize with such nucleotide sequences under stringent conditions or nucleic acid molecules which have sufficient homology to be a functional analogue of the nucleotide sequences mentioned, it may be determined whether the graft has a tendency towards non-physiological or pathological processes by itself or with respect to the receptor organism.

In another preferred embodiment of the invention, the rejection reaction, the course of a rejection and/or the rejection crisis is detected by an increased level of a nucleic acid molecule selected from the group consisting of:
a) a nucleic acid molecule comprising a nucleotide sequence selected from the group consisting of SEQ ID No. 1 and SEQ ID No. 2 or their complementary nucleotide sequences;
b) a nucleic acid molecule which will hybridize with a nucleotide sequence according to a) under stringent conditions;
c) a nucleic acid molecule comprising a nucleotide sequence which has sufficient homology with a nucleotide sequence according to a) or b) to be a functional analogue thereof;
d) a nucleic acid molecule which exhibits a genetic code degeneration relationship with respect to a nucleotide sequence according to any of a) to c); and
e) a nucleic acid molecule according to any nucleotide sequence of a) to d) which has been modified by deletions, additions, substitutions, translocations, inversions and/or insertions and is a functional analogue of a nucleotide sequence according to any of a) to d).

According to the invention, the nucleic acid molecules mentioned include, in particular, the nucleic acid molecules which will hybridize with the mentioned nucleic acid molecules under stringent conditions as well as those nucleic acid molecules which have sufficient homology to be a functional analogue of the nucleic acid molecules mentioned as well as those which exhibit a genetic code degeneration relationship or those which have been modified by deletions, additions, substitutions, translocations, inversions and/or insertions and are functional analogues of the mentioned nucleotide sequence of the nucleic acid molecules.

In another preferred embodiment of the invention, the tolerance or the course of a tolerance is detected by an increased level of a nucleic acid molecule selected from the group consisting of:
a) a nucleic acid molecule comprising a nucleotide sequence selected from the group consisting of SEQ ID No. 3, SEQ ID No. 4, SEQ ID No. 5, SEQ ID No. 6, SEQ ID No. 7 and SEQ ID No. 8 or their complementary nucleotide sequences;
b) a nucleic acid molecule which will hybridize with a nucleotide sequence according to a) under stringent conditions;
c) a nucleic acid molecule comprising a nucleotide sequence which has sufficient homology with a nucleotide sequence according to a) or b) to be a functional analogue thereof;
d) a nucleic acid molecule which exhibits a genetic code degeneration relationship with respect to a nucleotide sequence according to any of a) to c); and
e) a nucleic acid molecule according to any nucleotide sequence of a) to d) which has been modified by deletions, additions, substitutions, translocations, inversions and/or insertions and is a functional analogue of a nucleotide sequence according to any of a) to d).

Thus, advantageously, it is possible to determine by the detection of an increased level of the nucleic acid molecules mentioned whether the grafted organ, the grafted tissue of the single cell is being accepted by the receptor organism in such a way that pathological reactions are substantially absent.

The invention also relates to the use of the nucleic acid molecule, the vector, the host cell, the polypeptide, the recognition molecule and/or the vaccine in medical prophylaxis, clinical follow-up, graft follow-up treatment, clinical diagnostics and/or therapy. The skilled person can employ the nucleic acid molecules or vectors, host cells, polypeptides, recognition molecules and/or vaccines according to the invention in the fields of prophylaxis, follow-up, diagnostics or therapy. For example, it is possible to reduce the level of the biological structures which is increased in a rejection reaction or rejection crisis, in the form of a therapy to thereby enable, indicate or support a tolerance or conditions for a subsequent tolerance of the graft. This may be done, for example, by administering antisense constructs or RNA molecules which are capable of producing an RNA interference. However, it is also possible to functionally affect the peptides or proteins encoded by the nucleic acid molecules, whose level may also be increased, by antibodies in such a way that a physiological condition can be indicated, achieved or supported in the graft or between the graft and the receptor organism. Of course, it is also possible to increase a reduced level of nucleic acid molecules in the form of a therapeutic measure if a reduced level is associated with a rejection reaction or a rejection crisis. Various possibilities of modifying, especially increasing in the present case, the level of the mentioned substances or molecules are known to the skilled person. It is possible to increase the protein level, for example, by inserting an additional promoter upstream from the nucleic acid which encodes the corresponding protein, which may naturally occur in the organism or in the graft or is introduced into the grafted organ, or by enhancing the activity of the original promoter. Further, it is possible to increase the number of copies of the nucleic acids in the corresponding target tissue whereby more nucleic acid molecules are provided and more proteins can be expressed. The skilled person knows that such measures can be performed not only within the scope of a therapy, but also in a protocol for the prophylaxis or as a graft follow-up treatment. Clinical follow-ups and diagnostic measures can be advantageously effected in such a way that a quantification of the expression of the nucleic acid molecules or the peptides encoded by them or their fragments is effected at particular intervals, to be established by the skilled person, in the urine or biopsy material.

In another preferred embodiment of the invention, the nucleic acid molecules and their homologues or the modified nucleic acid molecules are used for the detection of T-cell-mediated immune processes, especially of pathogenic T-cell-mediated immune processes. The nucleic acid molecules according to the invention and also their derivatives, their complementary structures and the peptides encoded by them may be used, for example, for detecting complement reactions or other processes in which T cells have some importance. In particular, pathogenic T-cell-mediated immune processes, such as diabetes mellitus type I, rheumatoid arthritis, chronic bowel inflammation, dermatoses and/or allergies, can be detected.

In a particularly preferred embodiment of the invention, as T-cell-mediated immune processes, there are detected auto-immune diseases or inflammations, especially an antiglomerular basal membrane disease, auto-immune diseases of the nervous system, systemic lupus erythematosus, Addison's disease, antiphospholipid syndrome, IgA glomerulonephritis, Goodpasture's syndrome, Lambert-Eaton myasthenic syndrome, bullous pemphigoid, thrombocytopenic idiopathic purpura, auto-immune thyroiditis, rheumatoid arthritis, insulin-dependent diabetes mellitus, pemphigus, auto-immune hemolytic anemia, dermatitis herpetiformis Duhring, membranous glomerulonephritis, Graves' disease, sympathetic ophthalmia, auto-immune polyendocrinopathies, multiple sclerosis and/or Reiter's disease.

In another preferred embodiment of the invention, the T-cell-mediated immune processes are physiological, pathological and/or clinical graft reactions.

In another preferred embodiment of the invention, the graft reactions include a rejection crisis, a rejection reaction, a course of a rejection, a tolerance reaction and/or a course of a tolerance.

The invention also relates to a kit which comprises the nucleic acid molecule, the vector, the host cell, the polypeptide, the recognition molecule and/or the vaccine, and to the use of the kit for detecting the graft reaction.

The invention has a number of advantages. Thus, it is possible, in particular, to perform a permanent supervision of the condition of the graft after transplantations, it being possible to recover the nucleic acid molecules or peptides or recognition substances used as markers according to the invention from various samples from the patient, for example, urine. Thus, it is possible, in particular, to establish functional deteriorations of the graft early, say at the beginning of the effector chain. With the substances according to the invention and by the method according to the invention, it is also possible to make an early diagnosis of processes which are already proceeding subclinically. Thus, subclinically proceeding reactions need no longer be determined by control biopsies and conventional histology. Further, the substances mentioned can be used as markers for the monitoring of grafts in order to detect undesirable immune reactions before the organ is damaged, and with certainty in terms of differential diagnostics. The monitoring may be used, for example, as a follow-up in multiple immunosuppressant schemes, wherein one or more of the immunosuppressant components can be discontinued when the function is going well, wherein the occurrence of accelerated rejection processes can be recognized early. The method can also be optimized thereby individually from patient to patient. Also, using the markers, it is advantageously possible to transfer tolerance-induced protocols and tolerance-induced therapies to humans, since those in whom the therapy fails can be identified in due time after the discontinuation of the tolerance induction therapy in order to prevent irreversible damage to the graft. That is to say, the substances according to the invention, the method according to the invention and the uses provide exact analytical methods for evaluating the induction, the success and the retention of a tolerance. With the method according to the invention, a breakdown of the tolerance, for example, by the presence of an infection, can also be predicted, inter alia. Therefore, it is possible to make decisions about the safe discontinuation of an immunosuppressive therapy advantageously without having to risk the occurrence of rejection crises. Therefore, an important application of the nucleic acid according to the invention and the method according to the invention is the prediction of rejection crises during or after treatment, also after conventional therapies, before a deterioration of graft function occurs. Further, however, the conventional immunosuppression is also improved by the nucleic acid molecules according to the invention and the method according to the invention with respect to the early recognition of clinical and subclinical acute rejection crises and beginning chronic rejection processes. With the invention, it is possible to vary the therapy before the graft is detectably damaged. Further, it is possible to use the nucleic acid molecules according to the invention and the proteins or protein fragments encoded by them for the screening for drugs which can be employed in the diagnosis and therapy of graft reactions.

In the following, the invention shall be illustrated in more detail by Examples without being limited thereby.

### EXAMPLES

### Example 1

The nucleic acid molecules according to the invention can be identified in laboratory animals, for example, in the established orthotropic kidney transplantation model of rats in which the expression of the markers according to the invention can be used for post-surgical diagnostics. In the transplantation model employed (WF donor kidneys to BDIX receptors), several applications of an anti-CD4 antibody RIB5/2 can induce tolerance towards kidney grafts which are rejected between days 5 and 9 in the control antibody of treated receptor animals. The tolerance is characterized by a long lasting normal kidney function without an increase of the serum creatinine or proteinuria for more than 300 days. The infiltration of donor-reactive T cells is reduced only to 50%, but destruction of the grafted organ does not occur.

Within the scope of the invention, the mononuclear cells immigrated into the graft were isolated from receptor animals treated with control antibodies or RIB/2 on day 5 after the transplantation by collagen digestion and Fiscoll gradient, and their mRNA expression was compared by means of the “PCR select” method. This resulted in the isolation of cDNA fragments which are expressed at an increased level in grafts of rejecting receptor animals: 2A5 and 2A15 (corresponding to SEQ ID Nos. 1 and 2).

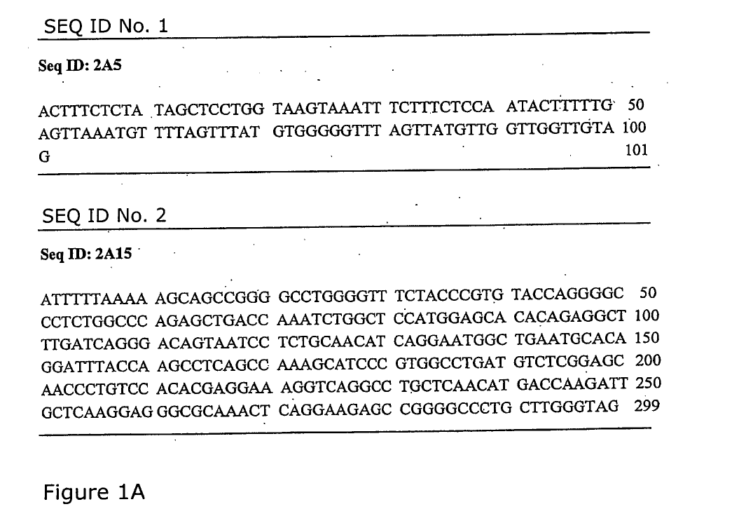

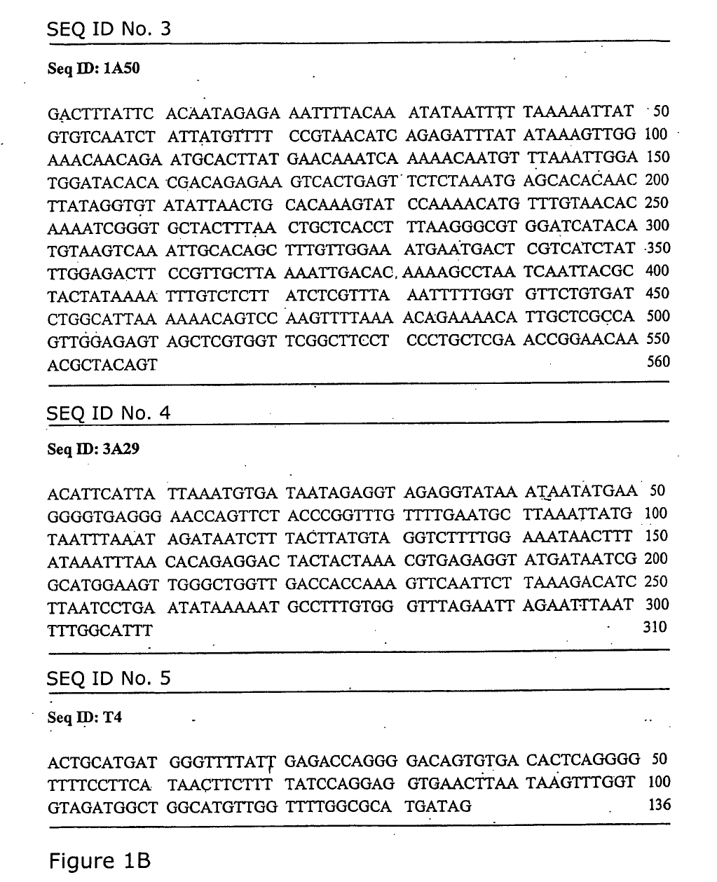

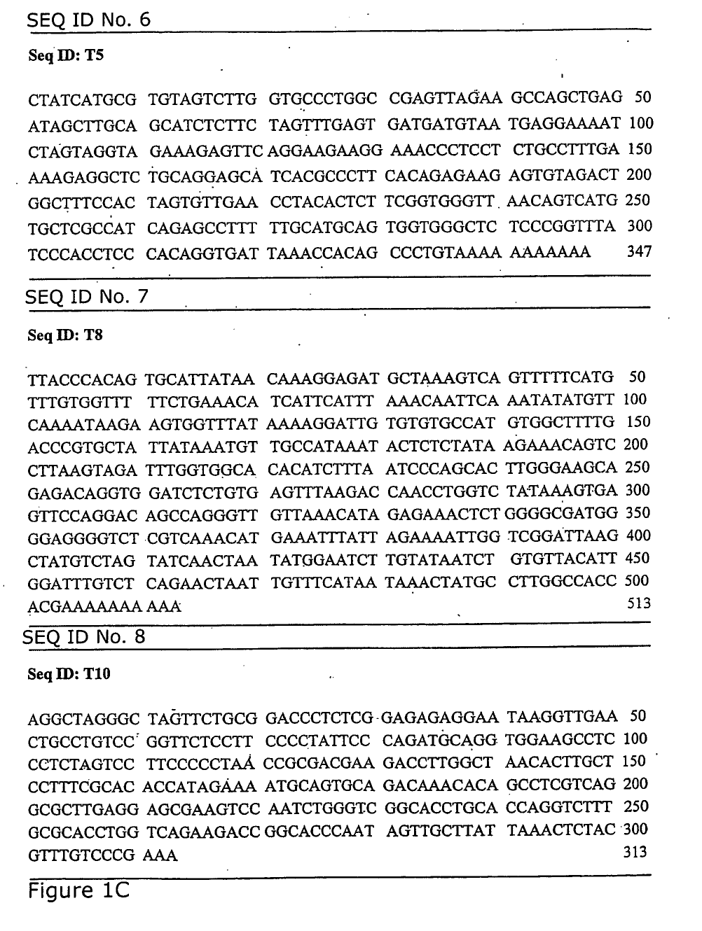

Also, cDNA fragments could be isolated whose expression is increased in grafts of tolerance-developing receptor animals: 1A50, 3A29, T4, T5, T8 and T10 (corresponding to SEQ ID Nos. 3, 4, 5, 6, 7 and 8). FIG. 1 shows the cDNA sequence segments of the fragments mentioned.

Further, according to the invention, oligonucleotide sequences for performing a real time RT PCR were derived from the sequence segments represented here. By means of these oligonucleotide sequences, a relative quantification of the expression of the corresponding mRNAs with respect to the “house-keeping gene” β-actin is possible in rat cells. Also, by using the homologous mouse sequences, oligonucleotide sequences were established for the relative quantification of the corresponding mRNAs with respect to the “house-keeping gene” HPRT in mouse cells.

Using the thus established oligonucleotide sequences, kinetic expression studies were performed in several transplantation models within the scope of the invention. In addition to the kidney transplantation model in rats as mentioned above, the expression of the fragments was also analyzed in a heart transplantation model in mice. In this model, a donor-specific blood transfusion (B10) is administered to the receptor animals (CBA) in combination with the anti-CD4 antibody YTS177 four weeks before the transplantation. This results in the induction of a donor-specific tolerance at the time of transplantation. Control hearts in untreated receptor animals are rejected between days 7 and 8.

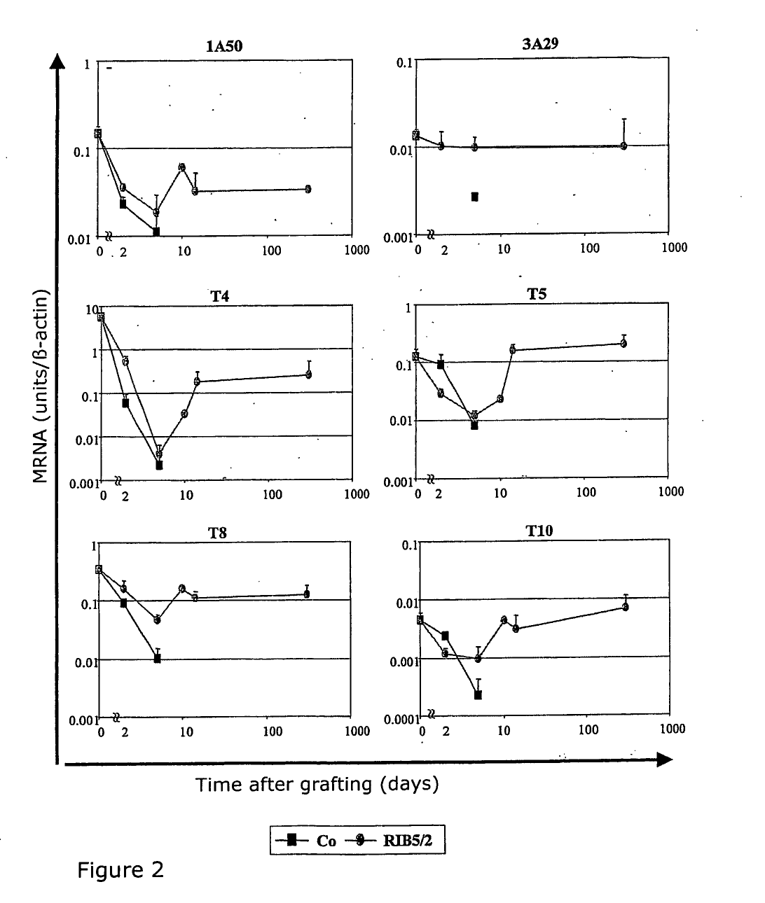

In FIG. 2, the results of the expression analysis are shown for the fragments 1A50, 3A29, T4, T5, T8 and T10 in the kidney transplantation model. There is shown the mRNA expression of the fragments within the graft for receptor animals treated with control antibodies (Co) on days 0 (naive kidneys), 2 and 5 after the transplantation, and in addition, there is shown the expression for RIB5/2-treated tolerance-developing receptor animals (RIB5/2) on days 0, 2, 5, 10, 14 and 300 after the transplantation. All cDNA fragments are strongly expressed in permanently accepted grafts, but in grafts of receptor animals treated with control antibodies, their expression is drastically decreased at the time of rejection.

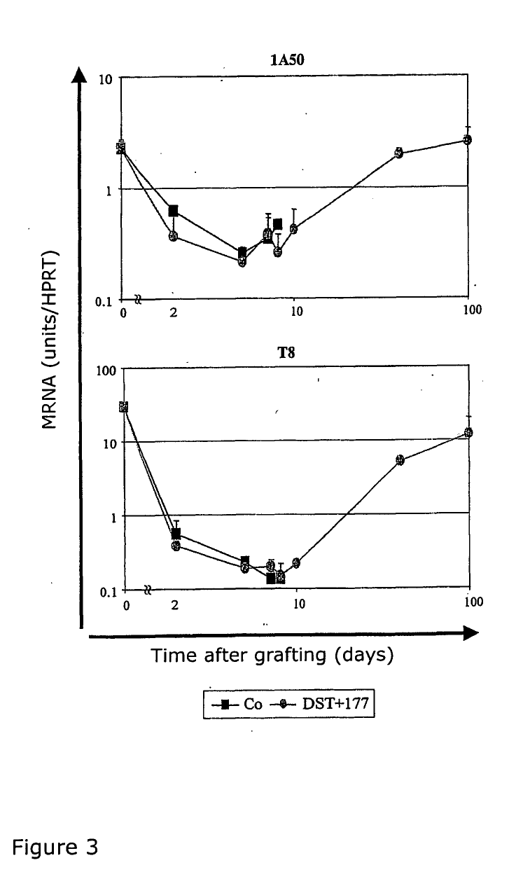

Subsequently, the expression of the corresponding mRNAs in the heart transplantation model was examined. In FIG. 3, the expression of the fragments 1A50 and T8 in the grafted organ is shown. The mRNA expression was analyzed in grafts of pretreated tolerance-developing receptor animals (DST+YTS177) on days 0 (naive hearts), 2, 5, 7, 8, 10, 40 and 100 after the transplantation. The results were compared with the mRNA expression in the graft on untreated control animals (Co) on days 0 (naive hearts), 2, 5, 7 and 8. In the heart transplantation model, permanently accepted grafts also exhibit a high mRNA expression of 1A50 and T8. In the grafts of rejecting receptor animals, the expression is again highly reduced.

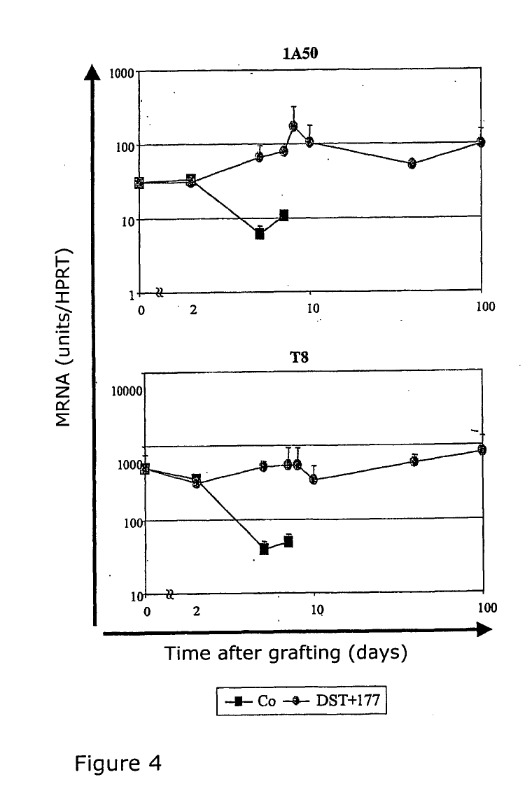

The different expression of 1A50 and T8 is also reflected in the peripheral blood. A drop of the expression of 1A50 and T8 shortly before the rejection (day 5) only occurs in the blood cells of untreated receptor animals (Co) (FIG. 4).

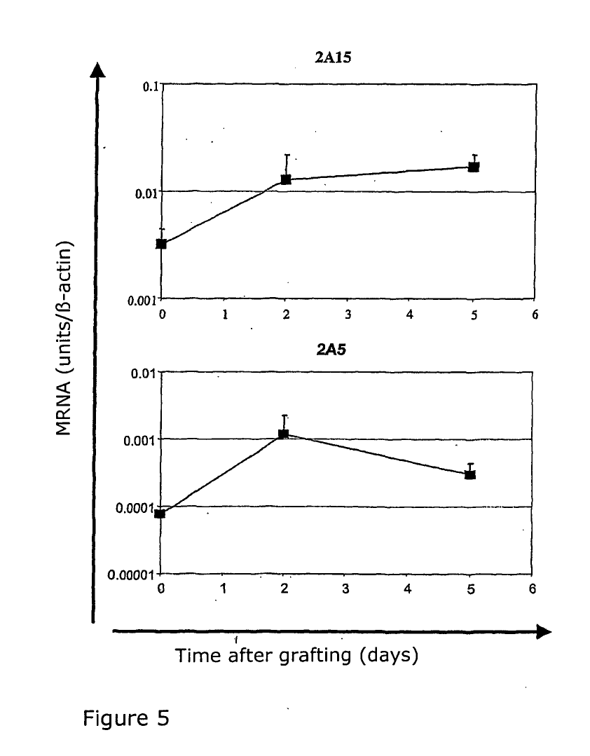

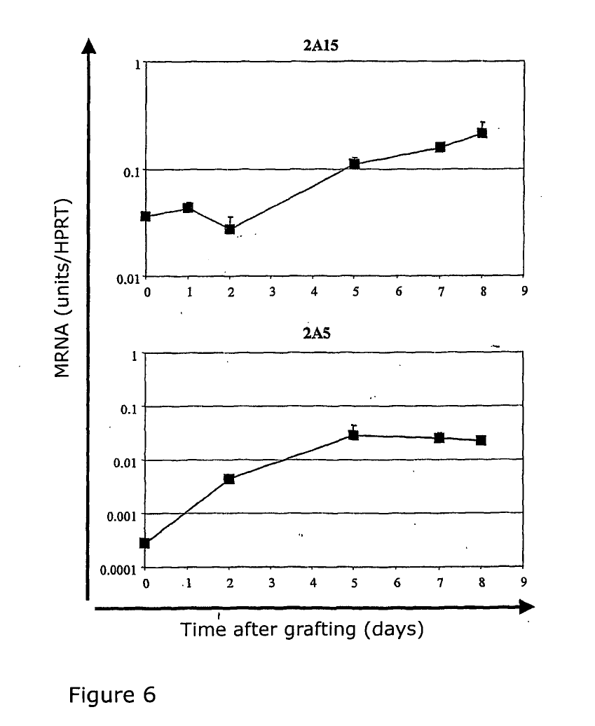

Further, the expression of the cDNA fragments 2A5 and 2A15 was examined in the kidney transplantation model (FIG. 5) and in the heart transplantation model (FIG. 6). The expression of these cDNA fragments in the graft of rejecting receptor animals is respectively shown. In both models, a high regulation of the mRNA expression occurs shortly before the rejection.

By means of the identification and quantification of such gene markers whose expression in the graft, in fluids from the graft (urine, lavage) or in peripheral blood correlates either with a long lasting good graft function or with the occurrence of rejections, a better evaluation of the tolerance-inducing therapy would be possible.

In the biopsy, the expression of 2A5 and 2A15 can be used for the evaluation of acute subclinical rejection crises and beginning chronic rejections. A strong and long lasting expression would be associated with the rejection of the organ. Only in a qualified way, this depends on the extent of infiltration of mononuclear cells into the graft, since the infiltration of mononuclear cells is only reduced to 50% in anti-CD4-treated tolerance-developing receptor animals in the kidney transplantation model. This would substantially improve the evaluation of a biopsy since not only infiltration into the organ is recurred to as a criterion of acute rejection, but also qualitative changes in the infiltrating cells. The expression of T4, T5, T10, 3A29, T8 and 1A50 in the biopsy can be recurred to, for example, for evaluating the success of a therapy. This would enable a decision about the safe discontinuation of the tolerance-inducing therapy.

The strong expression drop of 1A50 and T8 in the periphery in rejecting receptor animals more than 2 days before a clinical manifestation of rejection enables non-invasive diagnostics in the blood of the patient before a deterioration of the organ (e.g., increase of serum creatinine) can be detected.

The expression of 2A5 and 2A15 in the biopsy can be used for evaluating acute clinical and subclinical rejection crises and beginning chronic rejections. A strong and long lasting expression is associated with an immunological rejection of the organ. Only in a qualified way, this depends on the extent of infiltration of mononuclear cells into the graft, since the infiltration of mononuclear cells is only reduced to 50% in anti-CD4-treated tolerance-developing receptor animals in the kidney transplantation model. This substantially improves the evaluation of a biopsy since not only infiltration into the organ is recurred to as a criterion of acute rejection, but also the qualitative change of the infiltrating cells. The expression of T4, T5, T10, 3A29, T8 and 1A50 in the biopsy is recurred to for evaluating the success of the therapy. This enables a decision about the safe discontinuation of the tolerance-inducing therapy. The strong expression drop of 1A50 and T8 in the periphery in rejecting receptor animals more than 2 days before rejection enables non-invasive diagnostics in the blood or other body fluids, such as urine, of the patients before a deterioration of the organ, such as increase of serum creatinine, can be detected. Thus, the following diagnostic model after transplantation is successful:
1. Detection of 1A50 and T8 in the blood or other body fluids (e.g., urine) of the patient on a daily basis shortly after the operation and on a weekly/monthly basis in the further course for predicting a rejection crisis and thus failure of the therapy, and for detecting deficient suppression in discontinuation attempts before a deterioration of the organ can be detected.
2. Detection of 2A5 and 2A15 in control biopsies or graft-relevant body fluids (e.g., urine for kidney transplantation) in order to also detect rejection crises or deficient suppression in due time and to predict the risk of the development of a chronic rejection.
3. Detection of T4, T5, T8, T10, 1A50 and 3A29 in control biopsies or graft-relevant body fluids in order to appreciate the success of a tolerance-inducing or conventional therapy, in particular, in order to enable the risk-free discontinuation/reduction of the therapy.

### Example 2

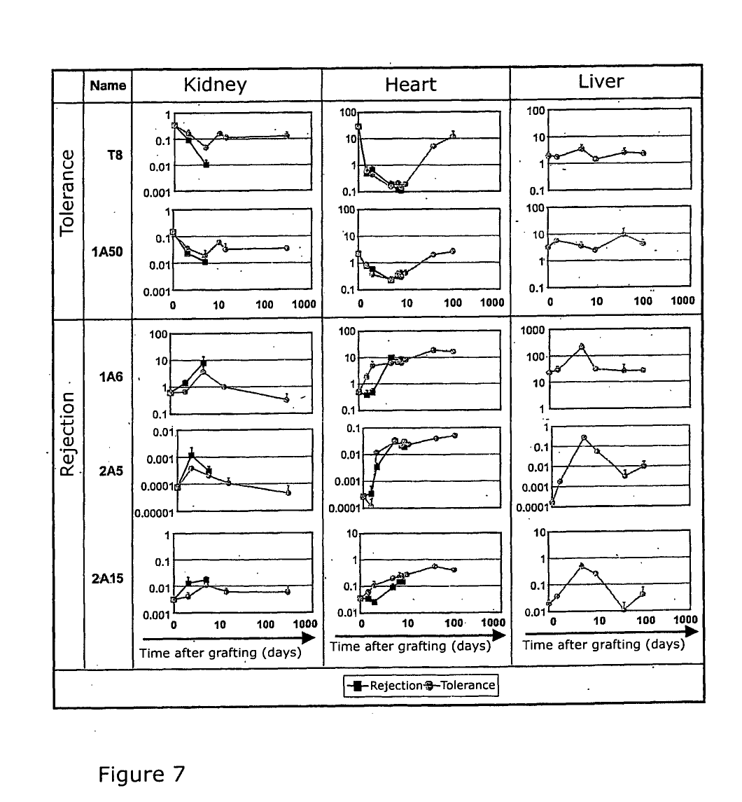

In another animal model for tolerance, biopsies were examined from mice which accept allogenic livers spontaneously, i.e., without being influenced by drugs (liver grafts from B10 mice to CBA receptor mice), i.e., develop a spontaneous tolerance, a phenomenon which may also occur after some years subsequent to an allogenic liver transplantation. On days 0, 1, 2, 5, 7, 8, 10, 40, 100 after the transplantation, the same markers were examined in the grafts as had been previously examined upon kidney or heart transplantations. FIG. 7 summarizes the results for comparison. The spontaneous tolerance with a transient self-limiting rejection crisis in this model is reflected by a stably high expression of the tolerance markers T8 and 1A50 over the whole observation period and a but temporary increase of the rejection markers 1A6, 2A5, 2A15 in the first week after the transplantation.

This points out the clear association of the expression of the mentioned markers with tolerance or rejection in another experimental model.

### Example 3

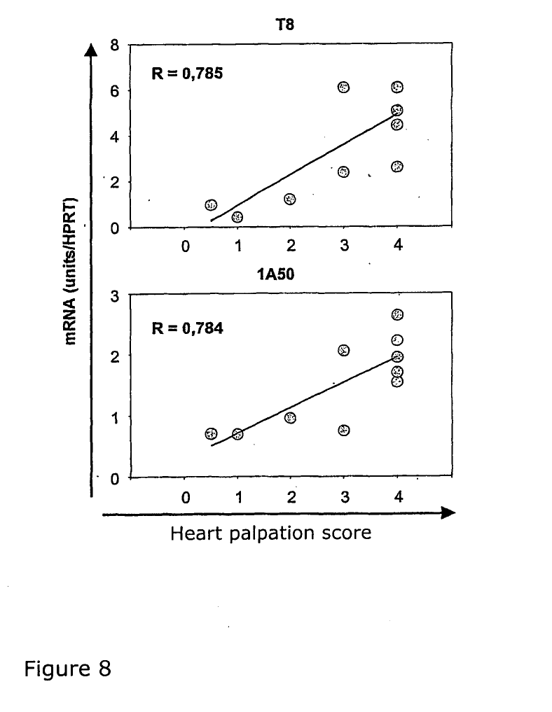

In the mouse heart transplantation model described, it could be shown that the majority of the hearts will survive on a long-term basis after tolerance induction with the protocol described, and that some hearts, however, develop signs of chronic rejection which are accompanied with a functional limitation. The latter can be determined by the “heart palpation score” (palpatory determination of the strength and rhythm of the heart beat), a high score (>3) indicating a good heart function. In a double-blind approach, the heart palpation score and expression of the tolerance markers T8 and 1A50 were determined in a comparative way and correlated with each other (FIG. 8), yielding a clear correlation between the functional ability of the heart and the expression of T8 (r=0.785) or 1A50 (r=0.784). This means that the two tolerance markers are very suitable for the prediction of incomplete tolerance, which, while not preventing acute rejection, prevents the development of a chronic rejection.

### Example 4

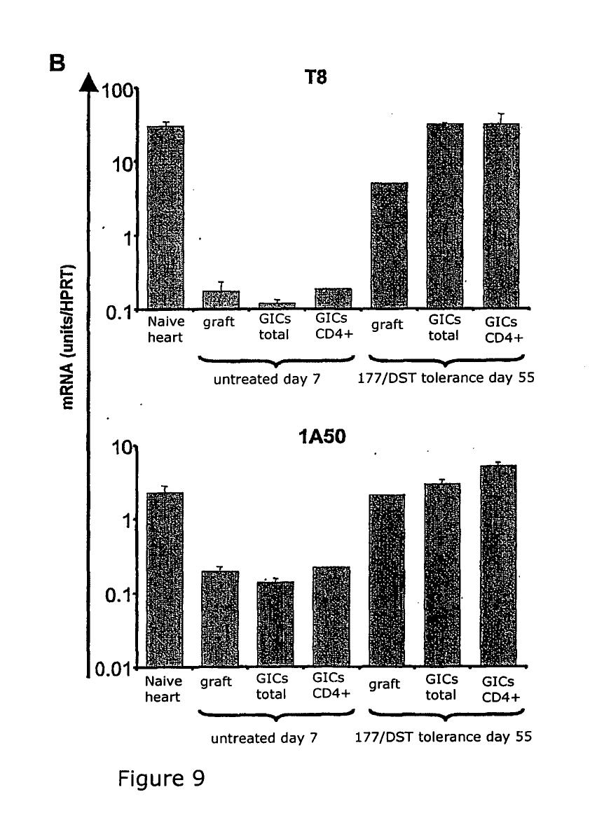

Numerous studies show that for maintaining a stable peripheral tolerance it is necessary to form specific regulatory CD4+ T cells which apparently accumulate in the tolerant graft where they inhibit the activation and effect of effector T cells. As has been published, tolerance can be transferred to naive animals also in our models with spleen cells and even more effectively with graft-infiltrating cells (GICs) (“infectious tolerance”). In order to verify whether the mentioned tolerance markers T8 and 1A50 are overexpressed in these cells, the GICs were isolated from the grafts by means of collagenase digestion, sorted using specific antibodies and characterized in terms of their gene expression. The data show that 1A50 and even more pronouncedly T8 is highly overexpressed in GICs from grafts of tolerant animals as compared to those from rejecting animals, and that such expression becomes detectable in sorted CD4+ GICs (FIG. 9). This suggests that graft-infiltrating regulatory T cells apparently express these tolerance markers.

### Example 5

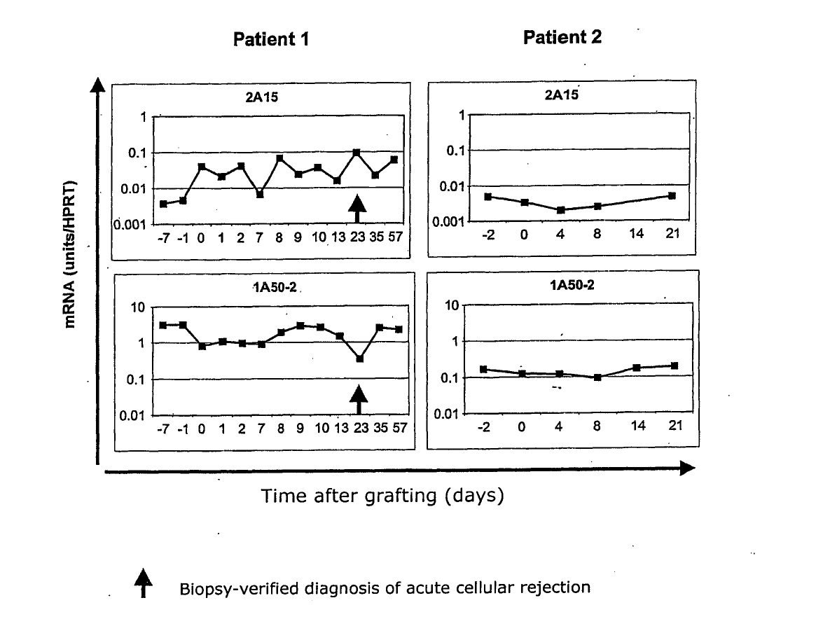

The human homologues of the sequences mentioned have been identified. Now, it was tested whether the markers can also be detected by means of real-time RT PCR in biopsies and blood leukocytes of kidney-grafted patients. 1A50, 2A5 and 2A15 could be detected in all biopsies and blood samples following kidney transplantation. Patient 1 developed an acute rejection on day 23 after transplantation from a live donor. At this time, a decrease of the tolerance marker 1A50 and an increase of the rejection marker 2A15 could be observed in the peripheral leukocytes (FIG. 10). Patient 2 showed a course without complications and hardly any variations in the expression of these markers.

Further, biopsies from the grafts of kidney-grafted patients were analyzed by means of real-time RT PCR. In biopsies, patients 3 and 4 showed signs of a subclinical rejection of Banff grades Ia and Ib, respectively. The tolerance marker 1A50 exhibited a relatively low expression for rejections (especially patient 4) as compared with biopsies from patients with stable functions and without signs of rejection in the graft (patients 5 and 6) (Table 1). In contrast, the expression of the rejection marker 2A15 was highest in the two samples with rejection (patients 3 and 4) and significantly lower for a normal function (patients 5 and 6). 2A5 was similarly detectable, but showed lesser differences.

Thus, the first data from patients confirm that the genes are also detectable in a human system, and that their regulation is very similar to that observed in the animal models.

TABLE 1
Gene expression in kidney biopsies from patients with grafts

| | | Gene expression | | |
|---|---|---|---|---|
| | | (in ratio to HPRT units) | | |
| | | 1A50 | 2A15 | 2A5 |
| Patient No. | Graft function | (×10⁻¹) | (×10⁻¹) | (×10⁻¹) |
| 3 | acute rejection | 3.0 | 5.2 | 0.2 |
| 4 | acute rejection | 1.8 | 4.0 | 0.2 |
| 5 | stable normal function | 5.5 | 0.4 | 0.1 |
| 6 | stable normal function | 7.9 | 0.2 | 0.1 |
| 7 | control kidney (no graft) | 4.0 | 0.2 | 0.1 |

What is claimed is

1-25. (canceled)

26. An isolated nucleic acid molecule selected from the group consisting of:
a) a nucleic acid molecule comprising a nucleotide sequence selected from the group consisting of SEQ ID Nos. 1 to 8 or their complementary nucleotide sequences;
b) a nucleic acid molecule which will hybridize with a nucleotide sequence according to a) under stringent conditions;
c) a nucleic acid molecule comprising a nucleotide sequence which has sufficient homology with a nucleotide sequence according to a) or b) to be a functional analogue thereof;
d) a nucleic acid molecule which exhibits a genetic code degeneration relationship with respect to a nucleotide sequence according to any of a) to c); and
e) a nucleic acid molecule according to any nucleotide sequence of a) to d) which has been modified by deletions, additions, substitutions, translocations, inversions and/or insertions and is a functional analogue of a nucleotide sequence according to any of a) to d).

27. The nucleic acid molecule according to claim 26, wherein the nucleotide sequence as stated under c) has at least 40% homology with one of the nucleotide sequences stated under a).

28. The nucleic acid molecule according to claim 26, wherein the nucleotide sequence as stated under c) has at least 60% homology with one of the nucleotide sequences stated under a).

29. The nucleic acid molecule according to claim 26, wherein the nucleotide sequence as stated under c) has at least 70% homology with one of the nucleotide sequences stated under a).

30. The nucleic acid molecule according to claim 26, wherein the nucleotide sequence as stated under c) has at least 80% homology with one of the nucleotide sequences stated under a).

31. The nucleic acid molecule according to claim 26, wherein the nucleotide sequence as stated under c) has at least 90% homology with one of the nucleotide sequences stated under a).

32. The nucleic acid molecule according to claim 26, wherein the nucleic acid molecule is at least one of genomic DNA, cDNA or RNA.

33. A vector comprising a nucleic acid molecule according to claim 26.

34. A host cell comprising the vector according to claim 33.

35. A polypeptide encoded by a nucleic acid molecule according to claim 26.

36. A recognition molecule directed against at least one of a nucleic acid molecule according to claim 26, a vector according to claim 33, a host cell according to claim 34 or a polypeptide according to claim 35.

37. The recognition molecule according to claim 36 being at least one of an antibody, an antibody fragment or an antisense construct.

38. The recognition molecule according to claim 36 being an RNA interference molecule.

39. A vaccine comprising at least one of a nucleic acid molecule according to claim 26, a vector according to claim 33, a host cell according to claim 34, a polypeptide according to claim 35, or a recognition molecule according to claims 36 or 37 or 38, optionally with a pharmaceutically acceptable carrier.

40. A method for the detection of graft reactions in a sample from a patient, characterized in that a level of at least one nucleic acid molecule according to claim 26 is determined in the sample, and the level is compared with a control level of a comparative sample from a healthy patient, wherein the graft reactions or the absence thereof (tolerance) are detected by a modified level in the sample as compared to the control level.

41. The method according to claim 40 wherein said graft is selected from at least one of lung, spleen, heart, kidney, liver, pancreas, or tissues.

42. The method according to claim 40, wherein said graft is selected from the group consisting of islets, aortas, or cartilage.

43. The method according to claim 40, wherein a DNA or RNA concentration, gene expression, number of copies of a nucleic acid, peptide concentration, peptide activity and/or as concentration of isoforms are determined as said level.

44. The method according to claim 40, wherein said level is determined as an mRNA concentration.

45. The method according to claim 40, wherein at least one of a rejection crisis, a rejection reaction, a course of a rejection, a tolerance reaction, or a course of a tolerance is detected as said graft reaction.

46. The method according to claim 33, wherein said rejection crisis, rejection reaction or course of a rejection is detected by a reduced level of a nucleic acid molecule comprising a nucleotide sequence selected from the group consisting of SEQ ID No. 3 and SEQ ID No. 7 or their complementary nucleotide sequences.

47. The method according to claim 33, wherein said rejection reaction, course of a rejection or rejection crisis is detected by an increased level of a nucleic acid molecule comprising a nucleotide sequence selected from the group consisting of SEQ ID No. 1 and SEQ ID No. 2 or their complementary nucleotide sequences.

48. The method according to claim 40, wherein said tolerance or course of a tolerance is detected by an increased level of a nucleic acid molecule comprising a nucleotide sequence selected from the group consisting of SEQ ID No. 3, SEQ ID No. 4, SEQ ID No. 5, SEQ ID No. 6, SEQ ID No. 7 and SEQ ID No. 8 or their complementary nucleotide sequences.

49. Use of a nucleic acid molecule according to claim 26, vector according to claim 33, host cell according to claim 34, polypeptide according to claim 35, recognition molecule according to claims 36 or 37 or 38 and/or vaccine according to claim 39 in at least one of medical prophylaxis, clinical follow-up, graft follow-up treatment, clinical diagnostics or therapy.

50. The use of the nucleic acid molecule according to claim 49 for the detection of T-cell-mediated immune processes.

51. The use of the nucleic acid molecule according to claim 49 for the detection of pathogenic T-cell-mediated immune processes.

52. The use according to claim 50, wherein said T-cell-mediated immune processes are auto-immune diseases or inflammations.

53. The use according to claim 50, wherein said T-cell-mediated immune processes are selected from the group consisting of an antiglomerular basal membrane disease, auto-immune diseases of the nervous system, systemic lupus erythematosus, Addison's disease, antiphospholipid syndrome, IgA glomerulonephritis, Goodpasture's syndrome, Lambert-Eaton myasthenic syndrome, bullous pemphigoid, thrombocytopenic idiopathic purpura, auto-immune thyroiditis, rheumatoid arthritis, insulin-dependent diabetes mellitus, pemphigus, auto-immune hemolytic anemia, dermatitis herpetiformis Duhring, membranous glomerulonephritis, Graves' disease, sympathetic ophthalmia, auto-immune polyendocrinopathies, multiple sclerosis and Reiter's disease.

54. The use according to claim 50, wherein said T-cell-mediated immune processes are at least one of physiological, pathological, clinical or subclinical graft reactions.

55. The use according to claim 54, wherein said graft reactions include at least one of a rejection crisis, a rejection reaction, a course of a rejection, a tolerance reaction or a course of a tolerance.

56. A kit comprising at least one of a nucleic acid molecule according to claim 26, a vector according to claim 33, a host cell according to claim 34, a polypeptide according to claim 35, a recognition molecule according to claims 36 or 37 or 38, or a vaccine according to claim 39.

57. Use of the kit according to claim 56 for the detection of a graft reaction.
